(19) United States
(12) Patent Application Publication
(10) Pub. No.: US 2022/0415463 A1
(43) Pub. Date: Dec. 29, 2022

(54) DRUG OVERDOSE PREVENTION SOFTWARE APPLICATION AND METHOD OF USE

(71) Applicant: Nicholas Metaxas, Bethal, CT (US)

(72) Inventor: Nicholas Metaxas, Bethal, CT (US)

(21) Appl. No.: 17/358,517

(22) Filed: Jun. 25, 2021

(51) Int. Cl. G16H 20/10; G16H 40/67; A61J 7/04; G06F 3/0481

(52) U.S. Cl. CPC G16H 20/10; G16H 40/67; A61J 7/0481; A61J 7/0472; A61J 7/0445; G06F 3/04817

(57) ABSTRACT

A drug overdose prevention software application and method of use that includes receiving a drug-using and a check-in time period and at least one emergency contact of a second user, generating on a user interface on the first user electronic computing device a drug using digital icon for indicating when the first user is about to use a drug, initiating an electronic timer for the drug-using time period upon modulation of the drug using digital icon, and generating a call-to-action digital icon on the user interface on the first user device and initiating an electronic timer for the check-in time period on the first user electronic computing device after the expiration of the drug-using time period. Thereafter, the method will either couple the first and second user electronic computing devices together over a network to notify the second user of the first user's failure to check-in or take no action.

### FIELD OF THE INVENTION

The present invention relates generally to software applications directed toward preventing drug or substance abuse overdoses and, more particularly, relates to software applications operably configured to ascertain whether a user has overdosed on drugs and notify others of said potential drug overdose.

### BACKGROUND OF THE INVENTION

Many users around the world struggle with drug addiction and/or substance abuse. These addictions and abuse can be harmful and even fatal for some users. One particularly harmful and/or lethal form of substance abuse includes a user introducing a drug, namely opioids, into the bloodstream of the user via a hollow hypodermic needle, which is pierced through the skin into the user's body (usually intravenously, but also at an intramuscular or subcutaneous location—collectively referred to as “IV”). Many opioid users are very well aware of the potential adverse health effects on the user from drug use, but said users are unable to stop using. Many of these users also care about their health and/or the emotional and health of others who care about the drug users. Conversely, many individuals (e.g., family, friends, etc.) also care about the health and safety and of a particular drug user.

Other than personally and constantly monitoring or observing a drug user, it almost impossible to know when a user is using drugs. To that end, some known systems and methods include attaching or otherwise coupling one or more physical monitoring device(s), e.g., sensors, to a user, wherein said monitoring device(s) ascertain one or more biological characteristic(s) of a user that may indicate the user is using drugs. Once the monitoring device(s) ascertain an indication or certain threshold level of a particular biological characteristic(s), the system communicatively coupled with the biological characteristic(s) may then notify others (family, friends, medical personnel, emergency responders, etc.) of said drug use and/or for intervention purposes. These systems and methods, however, have disadvantages. For example, many drug users will not voluntarily utilize or forget to utilize monitoring device(s). These monitoring device(s) are also prone to inadvertent dislodgement and false positives. Furthermore, many known systems and methods designed to ascertain drug overdose are often costly and impracticable for users.

Therefore, a need exists to overcome the problems with the prior art as discussed above.

### SUMMARY OF THE INVENTION

The invention provides a drug overdose prevention software application and method of use that overcomes the hereinafore-mentioned disadvantages of the heretofore-known devices and methods of this general type and that provides users a cost-efficient, effective, and less-intrusive system and process of preventing drug overdoses.

With the foregoing and other objects in view, there is provided, in accordance with the invention, a drug overdose prevention software application operably configured to be downloaded on a first user electronic computing device and a second user electronic computing device and that includes a computer-readable instructions programed to receive, from a first user, user-identifying information, a drug-using time period, a check-in time period, and at least one emergency contact of a second user, to generate a user interface on the first user electronic computing device with a drug using digital icon disposed thereon and for indicating when the first user is about to use a drug, to initiate an electronic timer for the drug-using time period upon modulation of the drug using digital icon, to generate a call-to-action digital icon on the user interface on the first user electronic computing device and initiate an electronic timer for the check-in time period on the first user electronic computing device after the expiration of the drug-using time period, to communicatively couple the first and second user electronic computing devices over a network after the expiration of the check-in time period and if the call-to-action digital icon is not manually modulated and generate an alert message on a user interface of the second user electronic computing device through the network, and to take no action if the call-to-action digital icon is manually modulated and regenerate the drug using digital icon on the user interface of the first user electronic computing device for indicating when the first user is about to use the drug.

In accordance with another feature, an embodiment of the present invention includes the computer-readable instructions programed to receive a selectively adjustable drug-using time period, and a selectively adjustable check-in time period.

In accordance with yet another feature, an embodiment of the present invention also includes the computer-readable instructions programed to receive the at least one emergency contact of the second user from a plurality of contacts digitally stored on a memory resident on the first user electronic computing device.

In accordance with an additional feature, an embodiment of the present invention also includes the computer-readable instructions programed to receive a customizable message from the drug user.

In accordance with a further feature of the present invention, the drug-using time period is greater than the check-in time period and the call-to-action digital icon is operably configured to be a slidable digital icon.

In accordance with a further feature, an embodiment of the present invention also includes the computer-readable instructions programed to generate a sensory alert on the first user electronic computing device during the check-in time period.

In accordance with a further feature of the present invention, the sensory alert is continuous during the check-in time period and includes at least one of an audio alert, visual alert, and vibrational alert through at least one transducer.

In accordance with an exemplary feature, an embodiment of the present invention also includes the check-in time period bifurcated into a first check-in time period and a second check-in time period and having the computer-readable instructions programed to receive, from the first user, the first check-in time period and the second check-in time period, to generate the call-to-action digital icon on the user interface on the first user electronic computing device, initiate the electronic timer for the first check-in time period on the first user electronic computing device after the expiration of the drug-using time period, and initiate the electronic timer for the second check-in time period on the first user electronic computing device after the expiration of the first check-in time period, to communicatively couple the first and second user electronic computing devices over a network after the expiration of the second check-in time period and if the call-to-action digital icon is not manually modulated, and to take no action if the call-to-action digital icon is manually modulated within either the first check-in time period or the second check-in time period and regenerate the drug using digital icon on the user interface on the first user electronic computing device for indicating when a user is about to use the drug.

In accordance with yet another embodiment, the present invention also includes the computer-readable instructions programed to receive, from a first user, a customizable text input at least partially forming the alert message and programmed to generate customized alert message on the user interface the second user electronic computing device through the network.

The present invention also discloses a user electronic computing device with an executable drug overdose prevention software application resident thereon and having a first user electronic computing device operably configured to execute computer-readable instructions programed to receive, from a first user, user-identifying information, a drug-using time period, a check-in time period, and at least one emergency contact of a second user, generate a user interface on the first user electronic computing device with a drug using digital icon disposed thereon and for indicating when the first user is about to use a drug, initiate an electronic timer for the drug-using time period upon modulation of the drug using digital icon, generate a call-to-action digital icon on the user interface on the first user electronic computing device and initiate an electronic timer for the check-in time period on the first user electronic computing device after the expiration of the drug-using time period, communicatively couple the first user electronic computing device with a second user electronic computing device over a network after the expiration of the check-in time period and if the call-to-action digital icon is not manually modulated and generate an alert message on a user interface the second user electronic computing device through the network, and to take no action if the call-to-action digital icon is manually modulated and regenerate the drug using digital icon on the user interface on the first user electronic computing device for indicating when the first user is about to use the drug.

Also in accordance with present invention, a computer-implemented process of preventing drug overdose is disclosed that includes the steps of providing a first user electronic computing device and a second user electronic computing device, the first user electronic computing device having a software application resident thereon, executing the software application on the first user electronic computing device, receiving, from a first user, user-identifying information, a drug-using time period, a check-in time period, and at least one emergency contact of a second user, generating on a user interface on the first user electronic computing device a drug using digital icon for indicating when the first user is about to use a drug, initiating an electronic timer for the drug-using time period upon modulation of the drug using digital icon, generating a call-to-action digital icon on the user interface on the first user electronic computing device and initiating an electronic timer for the check-in time period on the first user electronic computing device after the expiration of the drug-using time period, communicatively coupling the first and second user electronic computing devices over a network after the expiration of the check-in time period and if the call-to-action digital icon is not manually modulated and generating an alert message on a user interface of the second user electronic computing device through the network, and taking no action if the call-to-action digital icon is manually modulated and regenerate the drug using digital icon on the user interface of the first user electronic computing device for indicating when the first user is about to use the drug.

In accordance with yet another embodiment, the present invention also includes receiving, from the first user, a customizable text input at least partially forming the alert message and generating a customized alert message on the user interface the second user electronic computing device through the network.

In accordance with yet another embodiment, the present invention also includes receiving a selectively adjustable drug-using time period and a selectively adjustable check-in time period.

In accordance with a further feature of the present invention, the selectively adjustable drug-using time period is greater than the selectively adjustable check-in time period.

Although the invention is illustrated and described herein as embodied in a drug overdose prevention software application and method of use, it is, nevertheless, not intended to be limited to the details shown because various modifications and structural changes may be made therein without departing from the spirit of the invention and within the scope and range of equivalents of the claims. Additionally, well-known elements of exemplary embodiments of the invention will not be described in detail or will be omitted so as not to obscure the relevant details of the invention.

Other features that are considered as characteristic for the invention are set forth in the appended claims. As required, detailed embodiments of the present invention are disclosed herein; however, it is to be understood that the disclosed embodiments are merely exemplary of the invention, which can be embodied in various forms. Therefore, specific structural and functional details disclosed herein are not to be interpreted as limiting, but merely as a basis for the claims and as a representative basis for teaching one of ordinary skill in the art to variously employ the present invention in virtually any appropriately detailed structure. Further, the terms and phrases used herein are not intended to be limiting; but rather, to provide an understandable description of the invention. While the specification concludes with claims defining the features of the invention that are regarded as novel, it is believed that the invention will be better understood from a consideration of the following description in conjunction with the drawing figures, in which like reference numerals are carried forward. The figures of the drawings are not drawn to scale.

Before the present invention is disclosed and described, it is to be understood that the terminology used herein is for the purpose of describing particular embodiments only and is not intended to be limiting. The terms “a” or “an,” as used herein, are defined as one or more than one. The term “plurality,” as used herein, is defined as two or more than two. The term “another,” as used herein, is defined as at least a second or more. The terms “including” and/or “having,” as used herein, are defined as comprising (i.e., open language). The term “coupled,” as used herein, is defined as connected, although not necessarily directly, and not necessarily mechanically. The term “providing” is defined herein in its broadest sense, e.g., bringing/coming into physical existence, making available, and/or supplying to someone or something, in whole or in multiple parts at once or over a period of time. Also, for purposes of description herein, the terms “upper”, “lower”, “left,” “rear,” “right,” “front,” “vertical,” “horizontal,” and derivatives thereof relate to the invention as oriented in the figures and is not to be construed as limiting any feature to be a particular orientation, as said orientation may be changed based on the user's perspective of the device. Furthermore, there is no intention to be bound by any expressed or implied theory presented in the preceding technical field, background, brief summary or the following detailed description.

As used herein, the terms “about” or “approximately” apply to all numeric values, whether or not explicitly indicated. These terms generally refer to a range of numbers that one of skill in the art would consider equivalent to the recited values (i.e., having the same function or result). In many instances these terms may include numbers that are rounded to the nearest significant figure. In this document, the term “longitudinal” should be understood to mean in a direction corresponding to an elongated direction of the user interface of an electronic device or, if having no elongated direction, spanning from a top of the device to the bottom of the device. The terms “program,” “software application,” and the like as used herein, are defined as a sequence of instructions designed for execution on a computer system. A “program,” “computer program,” or “software application” may include a subroutine, a function, a procedure, an object method, an object implementation, an executable application, an applet, a servlet, a source code, an object code, a shared library/dynamic load library and/or other sequence of instructions designed for execution on a computer system.

### BRIEF DESCRIPTION OF THE DRAWINGS

The accompanying figures, where like reference numerals refer to identical or functionally similar elements throughout the separate views and which together with the detailed description below are incorporated in and form part of the specification, serve to further illustrate various embodiments and explain various principles and advantages all in accordance with the present invention.

FIG. 1 is an exemplary network implementing a drug overdose prevention software application in accordance with one embodiment of the present invention;

FIGS. 2-13 depict user interfaces on a first user electronic computing device in accordance with embodiments of the present invention;

FIG. 14 depicts a user interface on a second user electronic computing device in accordance with one embodiment of the present invention;

FIG. 15 depicts a user interface on a first user electronic computing device in accordance with one embodiment of the present invention;

FIGS. 16-20 depict user interfaces on a second user electronic computing device in accordance with embodiments of the present invention;

FIG. 21 is a block diagram depicting exemplary electrical components utilized in one or more of the user electronic computing devices in accordance with embodiments of the present invention; and

FIGS. 22a-b depict a process flow diagram depicting a computer-implemented process of preventing drug overdose in accordance with one embodiment of the present invention.

### DETAILED DESCRIPTION

While the specification concludes with claims defining the features of the invention that are regarded as novel, it is believed that the invention will be better understood from a consideration of the following description in conjunction with the drawing figures, in which like reference numerals are carried forward. It is to be understood that the disclosed embodiments are merely exemplary of the invention, which can be embodied in various forms.

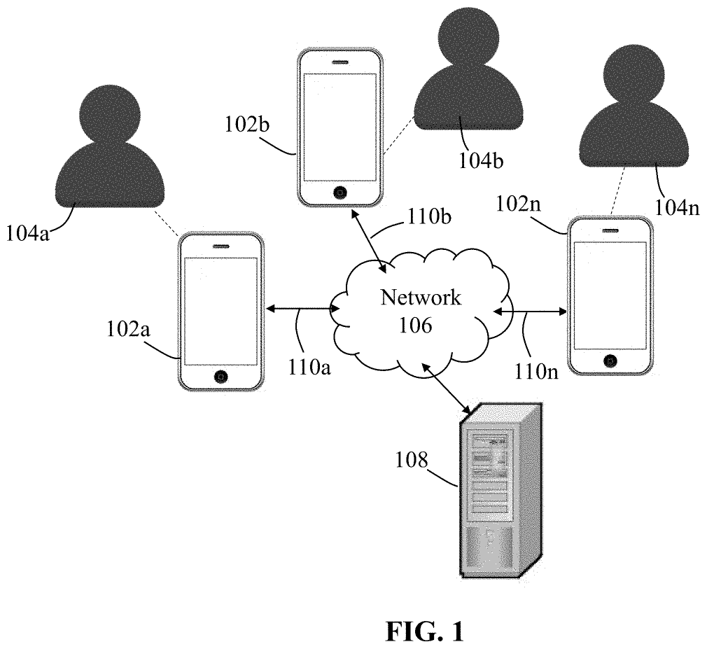

The present invention provides a novel and efficient drug overdose prevention software application and method of use that is cost-efficient and effective for many users with drug addiction and their family and other loved ones. Referring now to FIG. 1, one embodiment of the present invention implemented over a network 106 is shown in a schematic view. FIG. 1 along with the other figures depicted herein show several advantageous features of the present invention, but, as will be described below, the invention can be provided in several shapes, sizes, combinations of features and components, and varying numbers and functions of the components. The first example of a drug overdose prevention software application implemented over a network 106 is shown in FIG. 1. The invention requires at least two electronic computing devices 102a-n, wherein “n” represents any number greater than one and, as described further below, enables a drug user to notify other individuals or entities if the drug user has overdosed. As such, medical attention and other help may be provided to the drug user if the user does overdose on a drug.

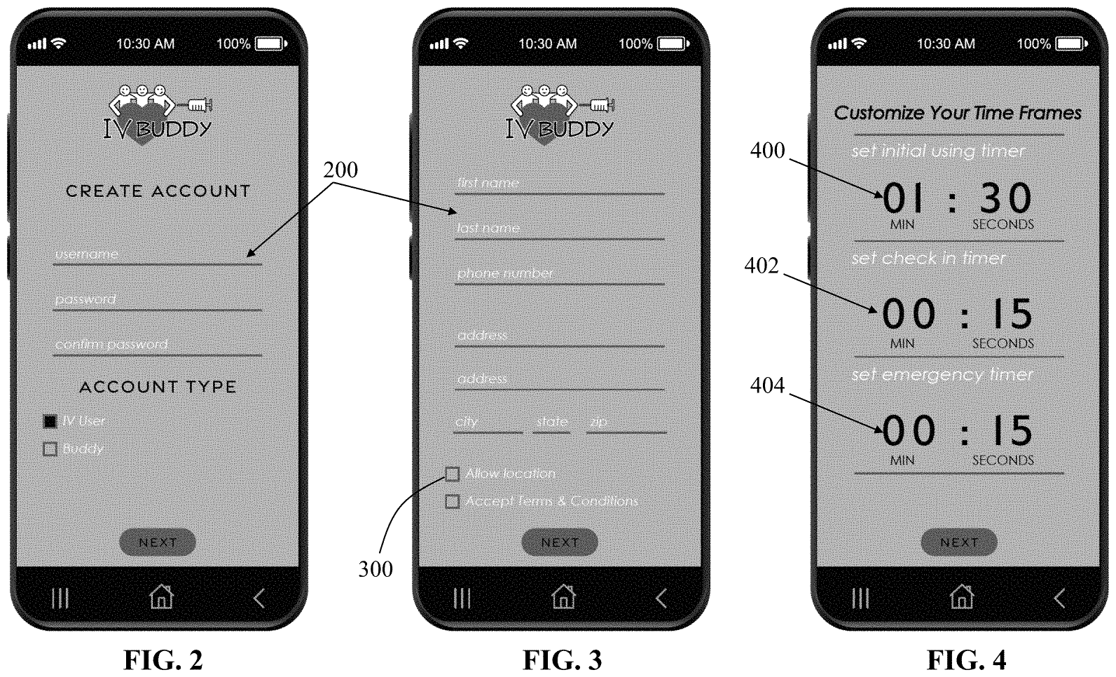

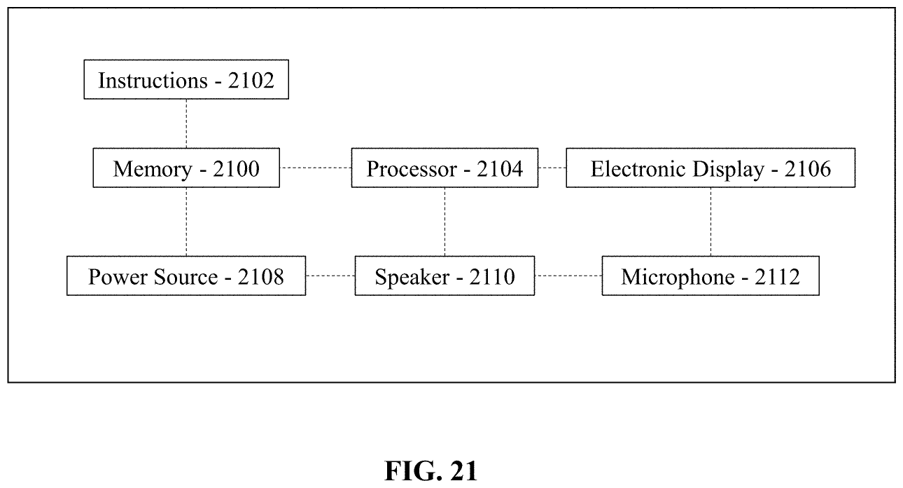

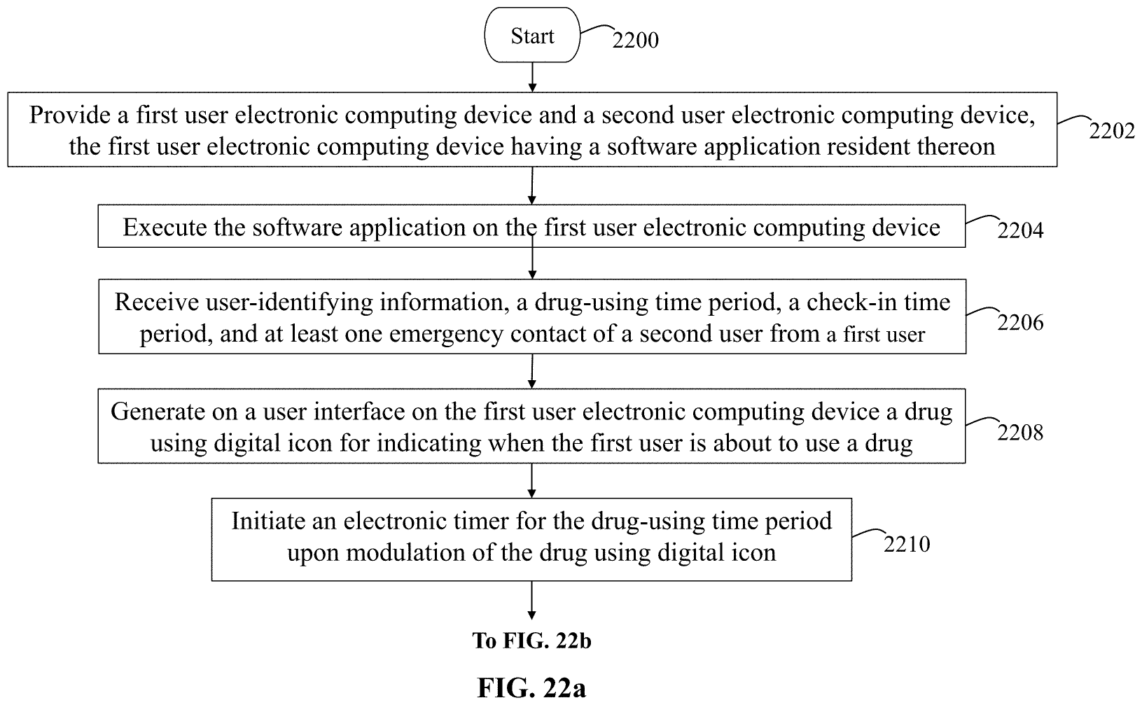

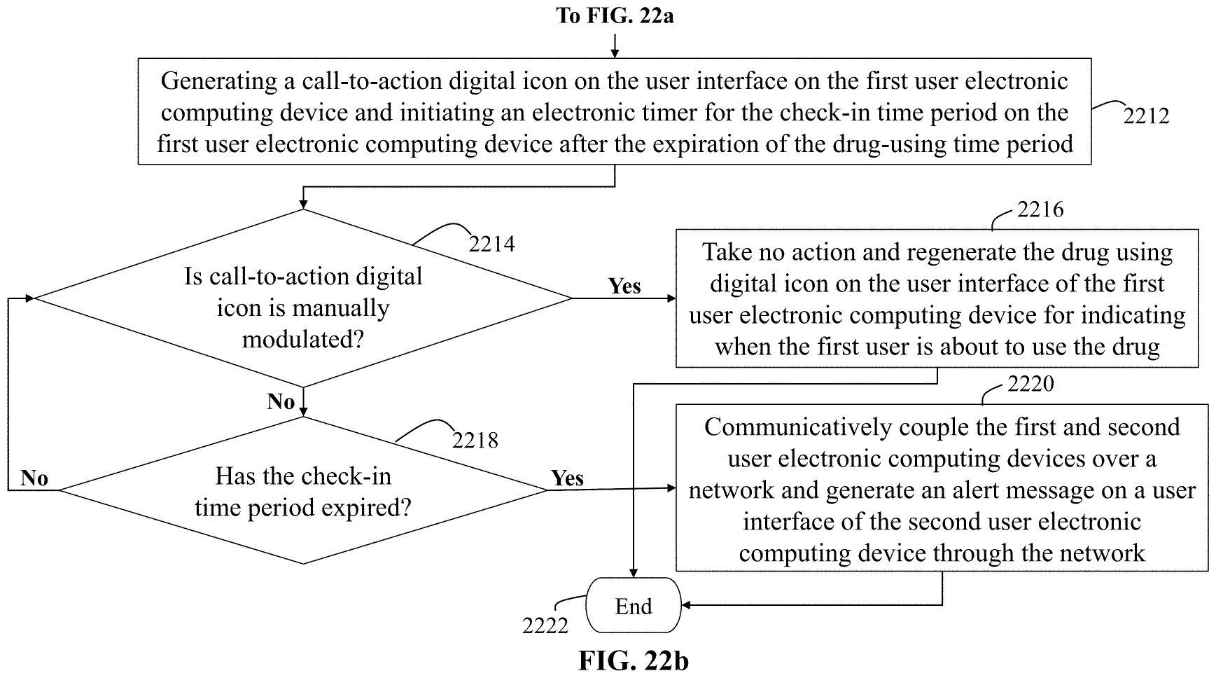

As seen in the figures, the software application is beneficially downloadable and downloaded onto the plurality of electronic computing devices 102a-n. The downloading may occur at separate times and may originate from an administrative server 108 that may also be operable to communicatively couple the devices 102a-n over the network 106. Although the electronic computing devices 102a-n are depicted as mobile phones, they may be other computing devices, such as tablets. In one embodiment, the software application is operably configured to be downloadable and/or downloaded on a first user electronic computing device 102a, e.g., the drug user, and a second user electronic computing device 102b, e.g., the drug user's family member. As best seen in FIGS. 2-7 and with reference to FIG. 21, the software application includes computer-readable instructions and/or a medium 2102 (e.g., written with objective-C, Swift, Laravel, Python, C++, etc.) programed to carry out steps implementing the present invention. As appreciated by the those of skill in the art, the instructions may be stored on the devices 102a-n and, when executed by a computer, cause the device to perform the inventive method described herein and exemplified in FIG. 22.

The software application may be first programmed to receive, from a first user 104a, user-identifying information 200 (e.g., name, phone number, account name, password, etc.), a drug-using time period 400 (e.g., selectively adjustable by the user or predefined), a check-in time period 402 (e.g., selectively adjustable by the user or predefined), and at least one emergency contact of a second user 104b. This data may be input manually through the user interface of the device 102a, through an auxiliary input, or dictated into a field by using a microphone. In one beneficial embodiment, the software application may also be programmed to display and receive the user's geographic location (both while using the software application and/or not using the software application). The user interface 700 may include a geographic indication icon 300 that confirms the drug user is allowing his or her geographic location to be tracked and/or conveyed to others. In another embodiment, the user-identifying information 200 may also include the address or location of the drug user 104a. The drug user's geographic location may be beneficially conveyed to others if and when an overdose is suspected based on the drug user's non-compliance with the call-to-action icon as describe below.

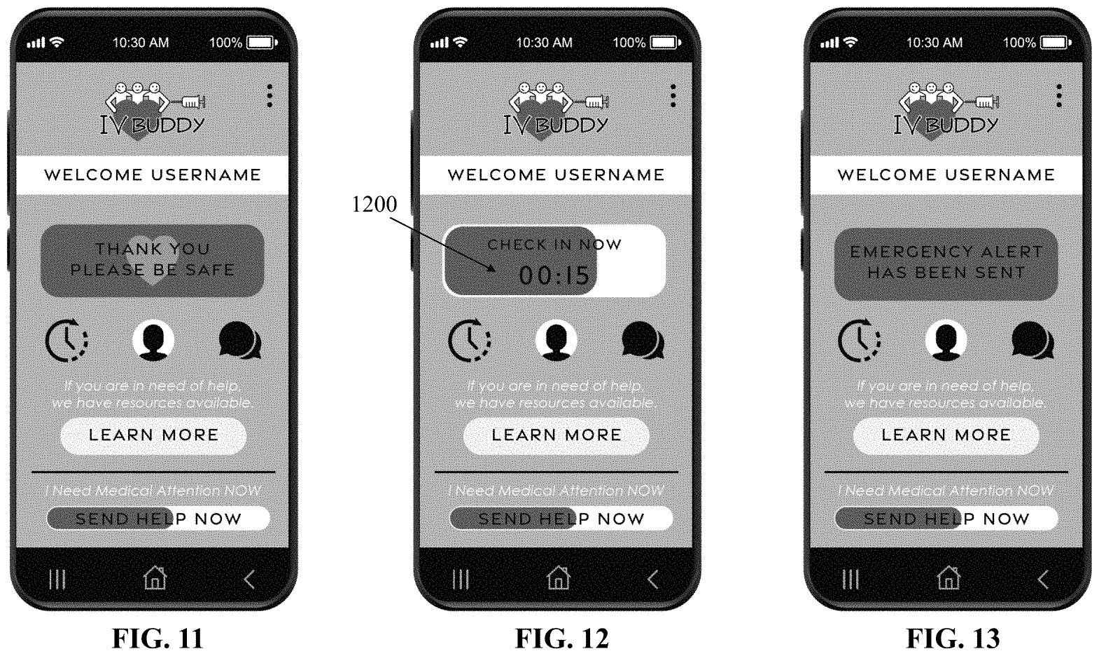

The user interface 700 for the first user's 104a device 102a may enable the user to provide and register user sign in information and providing set up the timing for each step in the sequence or use preloaded time frames, e.g., a drug-using time period 400 and a check-in time period 402 that may be bifurcated into a first check-in time period 402 and a second check-in time period 404. The computer-readable instructions 2102 may also be programed to receive the first check-in time period 402 and the second check-in time period 404 from the first user 104a. Said another way, the software application is operably configured to generate the call-to-action digital icon on the user interface 700 on the first user electronic computing device, initiate the electronic timer for the first check-in time period on the first user electronic computing device 102a after the expiration of the drug-using time period 400, and initiate the electronic timer for the second check-in time period on the first user electronic computing device 102a after the expiration of the first check-in time period. Further, the software application is operably configured to communicatively couple the first and second user electronic computing devices 102a-b over a network 106 after the expiration of the second check-in time period and if the call-to-action digital icon 1000 is not manually modulated. Similarly, the software application is configured to take no action if the call-to-action digital icon 1000 is manually modulated within either the first check-in time period or the second check-in time period and regenerate the drug using digital icon 702 on the user interface 700 on the first user electronic computing device 102a for indicating when a user is about to use the drug. FIG. 11 best depicts the software application taking no action, wherein the user interface may display a message confirming the user does not need help or for others to be notified. The second check-in time period 404 ensures the software application does not inadvertently contact another user 102b-n. In some embodiments, the second check-in time period 404 is the only period that provides a sensory alert. FIG. 12 best depicts the user interface displaying the electronic timer 1200 for the second check-in time period 404, wherein the electronic timer 1200 similarly counts down and is displayed if the first check-in time period 402 expires, i.e., the user 104a is given one last opportunity to modulate the call-to-action icon 1000 before the alert is sent out (which is best depicted in FIG. 13).

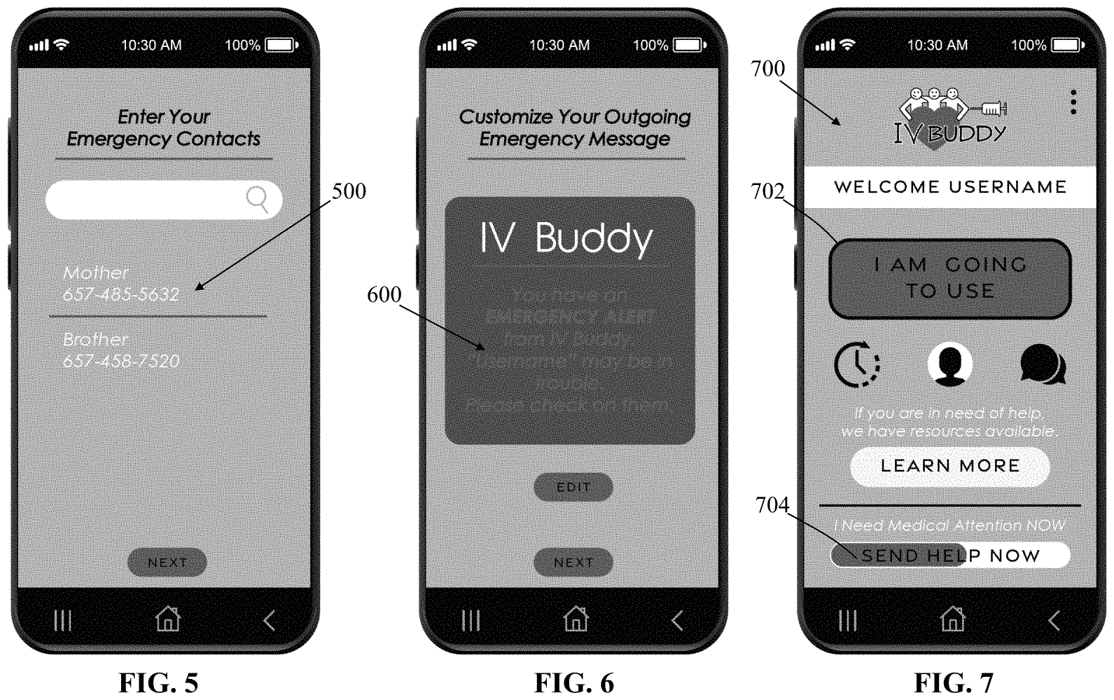

With respect to identifying other users 104b-n who may receive emergency notifications if the drug user 104a is unresponsive, the user interface 700 may provide (as best shown in FIG. 5) one or more fields for the user 104a to manually insert one or more emergency contacts. In other embodiments, the computer-readable instructions 2102 are programed to beneficially receive one or more emergency contact(s) of the second user(s) 104b-n from a plurality of contacts digitally stored on a memory 2100 resident on the first user electronic computing device 102a. In some embodiments, the software application will not all permit the user to move to the next step of registration unless the user 104a inserts at least two emergency contacts (e.g., family, friends, emergency responders, etc.). Next, as shown in FIG. 6, the software application may enable the user 104a to confirm or customize an outgoing alert message 600 that will be sent out to one or more other user(s) 104b-n. Said differently, the computer-readable instructions 2102 may be programed to receive a customizable message from the user 104a, e.g., the first user's name, username, address, geographic location, etc.

Specifically in one embodiment, the software application may also be programmed to generate a user interface 700 on the first user electronic computing device 102a with a drug using digital icon 702 disposed thereon and for indicating when the first user is about to use a drug. The drug using digital icon 702 beneficially enables physical contact or manipulation by the user and indicates when the user is about to use a drug that could lead to an overdose. At the time the drug using digital icon 702 is modulated (depressed, slid, or otherwise modifying or influencing), the software enables initiation of an electronic timer 704 that corresponds with or runs for the time period 400 input by the user or predetermined. The drug using digital icon 702 is conspicuously disposed on the user interface 700 after the user 104a registers or otherwise provides the identifying information. Additionally, the user interface may include an emergency help icon 704 that is programmed, when modulated by the user 104a, to notify emergency responders directly (e.g., by calling 911).

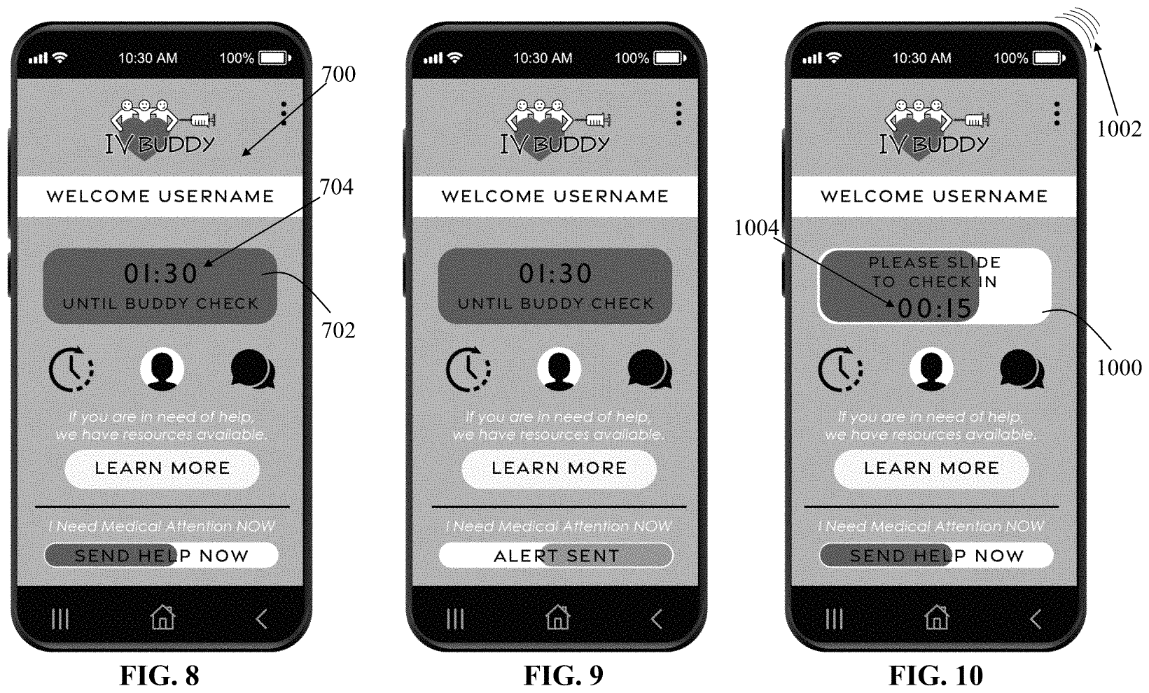

FIGS. 8-10 depict screenshots of the user interface 700 in an active timing sequence once the user modulates the drug using digital icon 702. Specifically, software application is operable to generate a call-to-action digital icon 1000 on the user interface 700 on the first user electronic computing device 102a and initiate an electronic timer 1004 for the check-in time period 402 on the first user electronic computing device 102a after the expiration of the drug-using time period 400. Exemplary time periods 400, 402, 404 are depicted in FIG. 4, but said time periods 400, 402, 404 may be predetermined or preprogrammed or, in other embodiments, selectively adjustable by the user 104a on the user interface 700 in accordance with the user's 104a desire. Said another way, the computer-readable instructions 2102 may be programed to receive a selectively adjustable drug-using time period 400 and a selectively adjustable check-in time period 402.

In preferred embodiments, the drug-using time period 400 is greater than the check-in time period 402 to prevent the generation of false positive alerts. In one embodiment, the call-to-action digital icon 1000 is operably configured to be a slidable digital icon that requires more modulation that normal icons, thereby ensuring the user 104a does not need help and/or avoid inadvertent notification to one or more other users 104b-n. Furthermore, the computer-readable instructions 2102 may be also programed to generate a sensory alert (e.g., vibration 1002) on the first user electronic computing device 102a during the check-in time period 402. The sensory alert may be continuously generated during the check-in time period 402 and may include one or more of the following: An audio alert, visual alert, and/or vibrational alert through at least one transducer on the device 102a (e.g., an electronic display 2106, a speaker 2110, a linear resonant actuator or other device to generate vibration).

If the user 104a does not manually modulate the call-to-action digital icon 1000 and after the expiration of the check-in time period 402 (whether if bifurcated or only having a single period), the software application is operably configured to communicatively couple the first and second user electronic computing devices 102a-b over a network 106. With reference to FIG. 1, the software application, devices 102a-n, system, and method may be operated over the network 106 and includes connections 110a-n, which are the medium used to provide communications links between various devices and computing device connected together. The connections 110a-n may be wired or wireless connections. A few exemplary wired connections are cable, phone line, and fiber optic. Exemplary wireless connections include radio frequency (RF) and infrared radiation (IR) transmission. Many other wired and wireless connections are known in the art and can be used with the present invention.

The network 106 may also include the Internet, which represents a worldwide collection of networks and gateways that use the TCP/IP suite of protocols to communicate with one another. At the heart of the Internet is a backbone of high-speed data communication lines between major nodes or host computers, consisting of thousands of commercial, government, educational and other computer systems that route data and messages. Of course, network 106 also may be implemented as a number of different types of networks, such as for example, an Intranet, a local area network (LAN), a wide area network (WAN), a cellular network. FIG. 1 is intended as an example, and not as an architectural limitation for the present invention. The network 106 may also include additional servers and other devices and entities not shown.

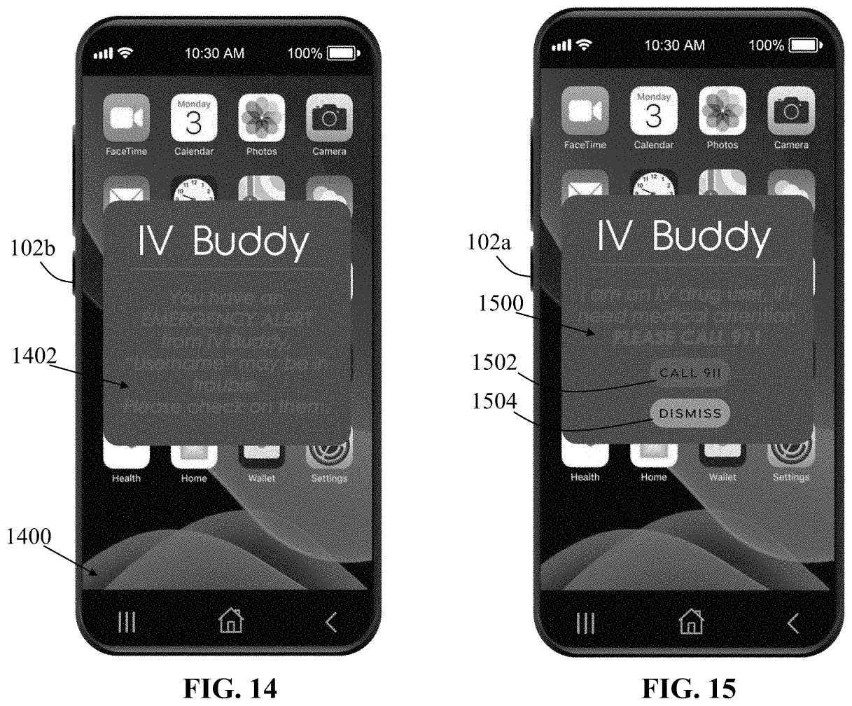

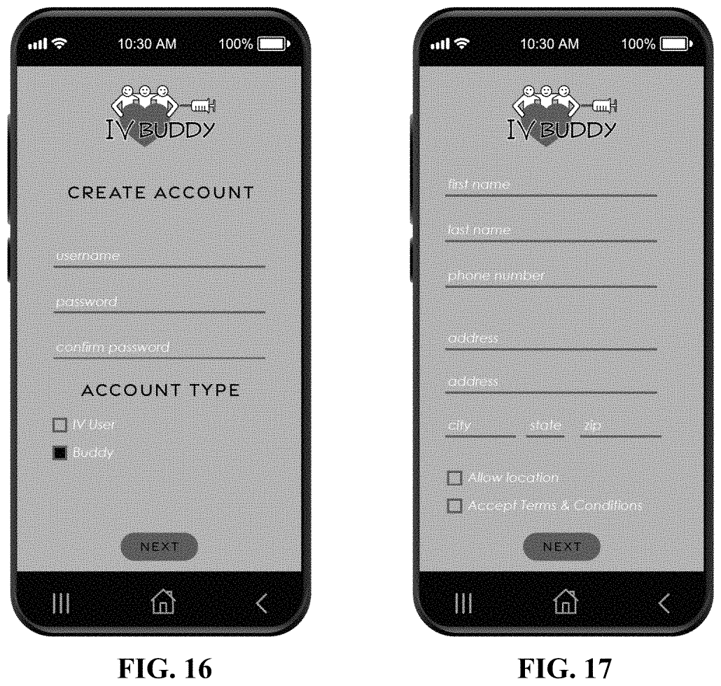

Referring to FIG. 1 with FIGS. 11-15, the software application is also operably configured to generate the alert message 600, 1402 on a user interface 1400 of the second user electronic computing device 102b through the network 106 once and if the user 104a does not modulate the call-to-action digital icon 1000. In one embodiment, the alert message 600, 1402 is communicated to the second electronic computing device 102b using “push” notifications (as is known in the art). Further, the software application is operably configured to cause an emergency or sensory alert (e.g., emergency tone that may bypass any audio volume settings—similar to an amber alert) on the second electronic computing device 102b. With reference specifically to FIG. 15, the first electronic computing device 102a may receive or otherwise cause to be displayed on the user interface (e.g., using a push notification) an alert message 1500 with interactive icons 1502, 1504 operably configured to contact emergency responders or dismiss the alert, respectively. As such, if another person is nearby the user who is overdosing, the nearby user or the overdosing user can effectively and quickly call 911 or dismiss the alert (if the user is not truly overdosing).

If the user does modulate the call-to-action digital icon 1000, the software application will be programmed to take no action (best exemplified in FIG. 11). Said another way, the software application will be programmed to take no action if the call-to-action digital icon 1000 is manually modulated and is operable to regenerate the drug using digital icon 702 on the user interface 700 of the first user electronic computing device 102a for indicating when the first user is about to use the drug (thereby repeating the timing process).

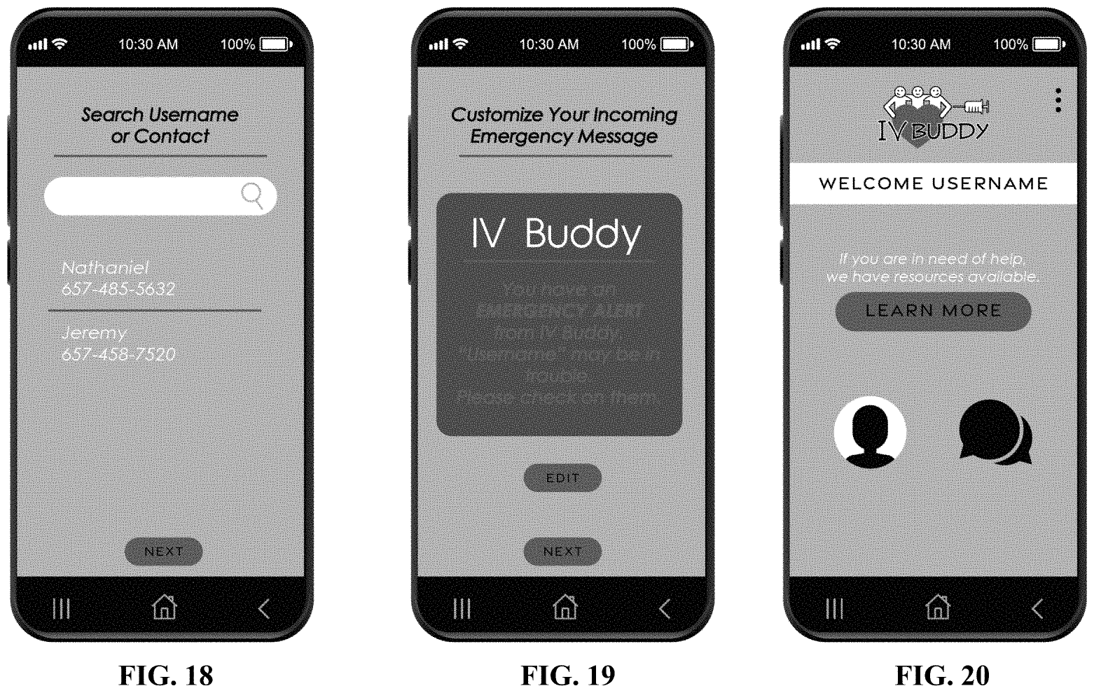

FIGS. 16-20 depict screenshots of the user 104b who can register with the software application, connect with other users, and receive alerts (as depicted therein).

FIG. 21 depicts a schematic block diagram of electrical components that may be coupled to or otherwise included on the devices 102a-n and one or more servers 108. Some of these components includes a non-transitory memory 2100 having the instructions 2102 stored thereon, a processor 2104 for implementing the instructions, an electronic display 2106, a power source 2108 (e.g., battery) providing power to the electrical components utilized in the device, and a speaker 2110, microphone 2112, or other transducers. Other components, however, may be utilized.

FIGS. 1-20 will now be described in conjunction with the process flow chart of FIGS. 22a-b. Although FIGS. 22a-b show a specific order of executing the process steps, the order of executing the steps may be changed relative to the order shown in certain embodiments. Also, two or more blocks shown in succession may be executed concurrently or with partial concurrence in some embodiments. Certain steps may also be omitted in FIGS. 22a-b for the sake of brevity. In some embodiments, some or all of the process steps included in FIGS. 22a-b can be combined into a single process. Specifically, a computer-implemented process of preventing drug overdose begins at step 2200 and immediately proceeds to step 2202, which includes providing a first user electronic computing device 102a and a second user electronic computing device 102b, wherein the first user electronic computing device 102a having a software application resident thereon. Next, the process includes the step 2204 of executing the software application on the first user electronic computing device 102a and then proceeds to step 2206 of receiving, from a first user 104a, user-identifying information 200, a drug-using time period 400, a check-in time period 402, and at least one emergency contact of a second user 104b.

Next, step 2208 includes generating on a user interface 700 on the first user electronic computing device 102a a drug using digital icon 702 for indicating when the first user is about to use a drug. Step 2210 includes initiating an electronic timer 704 for the drug-using time period 400 upon modulation of the drug using digital icon 702. Thereafter, step 2212 includes generating a call-to-action digital icon on the user interface 700 on the first user electronic computing device 102a and initiating an electronic timer 1004 for the check-in time period 402 on the first user electronic computing device 102a after the expiration of the drug-using time period 400. The process may then continue to a query step 2214, wherein the program will ascertain whether the call-to-action digital icon 1000 has been modulated. If the answer to that query is yes, then the process proceeds to step 2216 and the program will take no action and regenerate the drug using digital icon on the user interface of the first user electronic computing device for indicating when the first user is about to use the drug. If the answer to that query is no, then the process proceeds to another query step 2218 and the program will ascertain if the check-in time period expired. If no, then the process will revert back to the query step 2214 until the check-in time period expires. If the answer to the query step 2218 is yes, then the process continues to step 2220 of communicatively coupling the first and second user electronic computing devices over a network and generate an alert message on a user interface of the second user electronic computing device through the network. The process may terminate at step 2222 after step 2216 or step 2220.

A drug overdose prevention software application operably configured to be downloaded on a first user electronic computing device and a second user electronic computing device has been disclosed that is operable to keep drug users, namely IV drug users, safe by bringing together a dependable support network. Other users, or “buddies,” can be linked to users accounts to be notified when a drug user is using and may need medical attention. Said differently, overdoses are common, but more deadly with IV drug users, wherein the present invention is designed to prevent overdoses from becoming lethal. To that end, the drug overdose prevention software application will enable an active drug user to activate a timing sequence when they are about to use and if they do not check back into the software application within a particular period of time, the buddies are notified and are able to contact the proper authorities or otherwise get help of the drug user.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the above described features.

What is claimed is:

1. A drug overdose prevention software application operably configured to be downloaded on a first user electronic computing device and a second user electronic computing device and comprising:
computer-readable instructions programed to:
receive, from a first user, user-identifying information, a drug-using time period, a check-in time period, and at least one emergency contact of a second user;
generate a user interface on the first user electronic computing device with a drug using digital icon disposed thereon and for indicating when the first user is about to use a drug;
initiate an electronic timer for the drug-using time period upon modulation of the drug using digital icon;
generate a call-to-action digital icon on the user interface on the first user electronic computing device and initiate an electronic timer for the check-in time period on the first user electronic computing device after the expiration of the drug-using time period;
communicatively couple the first and second user electronic computing devices over a network after the expiration of the check-in time period and if the call-to-action digital icon is not manually modulated and generate an alert message on a user interface of the second user electronic computing device through the network; and
take no action if the call-to-action digital icon is manually modulated and regenerate the drug using digital icon on the user interface of the first user electronic computing device for indicating when the first user is about to use the drug.

2. The drug overdose prevention software application according to claim 1, further comprising:
computer-readable instructions programed to receive a selectively adjustable drug-using time period, and a selectively adjustable check-in time period.

3. The drug overdose prevention software application according to claim 1, further comprising:
computer-readable instructions programed to receive the at least one emergency contact of the second user from a plurality of contacts digitally stored on a memory resident on the first user electronic computing device.

4. The drug overdose prevention software application according to claim 1, further comprising:
computer-readable instructions programed to receive a customizable message.

5. The drug overdose prevention software application according to claim 1, wherein:
the drug-using time period is greater than the check-in time period.

6. The drug overdose prevention software application according to claim 1, wherein:
the call-to-action digital icon is operably configured to be a slidable digital icon. The drug overdose prevention software application according to claim 1, further comprising:
computer-readable instructions programed to generate a sensory alert on the first user electronic computing device during the check-in time period.

8. The drug overdose prevention software application according to claim 1, wherein:
the sensory alert is continuous during the check-in time period and includes at least one of an audio alert, visual alert, and vibrational alert through at least one transducer.

9. The drug overdose prevention software application according to claim 1, wherein the check-in time period is bifurcated into a first check-in time period and a second check-in time period, further comprising:
computer-readable instructions programed to:
receive, from the first user, the first check-in time period and the second check-in time period;
generate the call-to-action digital icon on the user interface on the first user electronic computing device, initiate the electronic timer for the first check-in time period on the first user electronic computing device after the expiration of the drug-using time period, and initiate the electronic timer for the second check-in time period on the first user electronic computing device after the expiration of the first check-in time period;
communicatively couple the first and second user electronic computing devices over a network after the expiration of the second check-in time period and if the call-to-action digital icon is not manually modulated; and
take no action if the call-to-action digital icon is manually modulated within either the first check-in time period or the second check-in time period and regenerate the drug using digital icon on the user interface on the first user electronic computing device for indicating when a user is about to use the drug.

10. The drug overdose prevention software application according to claim 1, further comprising:
computer-readable instructions programed to receive, from a first user, a customizable text input at least partially forming the alert message and programmed to generate customized alert message on the user interface the second user electronic computing device through the network.

11. A user electronic computing device with an executable drug overdose prevention software application resident thereon and comprising:
a first user electronic computing device operably configured to execute computer-readable instructions programed to:
receive, from a first user, user-identifying information, a drug-using time period, a check-in time period, and at least one emergency contact of a second user;
generate a user interface on the first user electronic computing device with a drug using digital icon disposed thereon and for indicating when the first user is about to use a drug;
initiate an electronic timer for the drug-using time period upon modulation of the drug using digital icon;
generate a call-to-action digital icon on the user interface on the first user electronic computing device and initiate an electronic timer for the check-in time period on the first user electronic computing device after the expiration of the drug-using time period;
communicatively couple the first user electronic computing device with a second user electronic computing device over a network after the expiration of the check-in time period and if the call-to-action digital icon is not manually modulated and generate an alert message on a user interface the second user electronic computing device through the network; and
take no action if the call-to-action digital icon is manually modulated and regenerate the drug using digital icon on the user interface on the first user electronic computing device for indicating when the first user is about to use the drug.

12. The user electronic computing device according to claim 11, further comprising:
computer-readable instructions programed to receive, from the first user, a customizable text input at least partially forming the alert message and programmed to generate customized alert message on the user interface the second user electronic computing device through the network.

13. The drug overdose prevention software application according to claim 11, further comprising:
computer-readable instructions programed to receive a selectively adjustable drug-using time period, and a selectively adjustable check-in time period.

14. The user electronic computing device according to claim 13, wherein:
the selectively adjustable drug-using time period is greater than the selectively adjustable check-in time period.

15. A computer-implemented process of preventing drug overdose comprising the steps of:
providing a first user electronic computing device and a second user electronic computing device, the first user electronic computing device having a software application resident thereon;
executing the software application on the first user electronic computing device;
receiving, from a first user, user-identifying information, a drug-using time period, a check-in time period, and at least one emergency contact of a second user;
generating on a user interface on the first user electronic computing device a drug using digital icon for indicating when the first user is about to use a drug;
initiating an electronic timer for the drug-using time period upon modulation of the drug using digital icon;
generating a call-to-action digital icon on the user interface on the first user electronic computing device and initiating an electronic timer for the check-in time period on the first user electronic computing device after the expiration of the drug-using time period;
communicatively coupling the first and second user electronic computing devices over a network after the expiration of the check-in time period and if the call-to-action digital icon is not manually modulated and generating an alert message on a user interface of the second user electronic computing device through the network; and
taking no action if the call-to-action digital icon is manually modulated and regenerate the drug using digital icon on the user interface of the first user electronic computing device for indicating when the first user is about to use the drug.

16. The method according to claim 15, further comprising:
receiving, from the first user, a customizable text input at least partially forming the alert message and generating a customized alert message on the user interface the second user electronic computing device through the network 106.

17. The method according to claim 15, further comprising:
receiving a selectively adjustable drug-using time period and a selectively adjustable check-in time period.

18. The method according to claim 15, wherein:
the selectively adjustable drug-using time period is greater than the selectively adjustable check-in time period.
